(19) United States
(12) Patent Application Publication
(10) Pub. No.: US 2018/0338856 A1
(43) Pub. Date: Nov. 29, 2018

(54) SYSTEMS, METHODS AND ORAL APPLIANCE DEVICES

(71) Applicant: Diamond Orthotic Laboratory, La Mesa, CA (US)

(72) Inventors: Matthew M. Rago, San Diego, CA (US); Steven R. Olmos, Alpine, CA (US)

(73) Assignee: Diamond Orthotic Laboratory, La Mesa, CA (US)

(21) Appl. No.: 15/990,544

(22) Filed: May 25, 2018

Related U.S. Application Data
(60) Provisional application No. 62/511,902, filed on May 26, 2017.

(51) Int. Cl. A61F 5/56; A61F 5/08

(52) U.S. Cl. CPC A61F 5/566; A61C 7/36; A61F 5/08

(57) ABSTRACT

Systems, methods are provided for creating and using oral appliances. These oral appliances can be inserted into a patient's mouth to reduce or remove resistance in a patient's airway by controlling biological aspects of nasal and tongue positioning and movement. As such, these appliances can improve anatomic and functional relationships of the oropharyngeal area.

This patent application claims priority to U.S. Provisional Patent Application No. 62/511,902 filed on May 26, 2017, which is incorporated by reference herein, in its entirety.

### BACKGROUND OF THE SYSTEM

The subject matter described herein relates generally to mandibular oral appliance devices and associated systems and methods that are designed to control the tongue, reduce airway collapse, and aid air flow through both the nasal and posterior pharyngeal regions of the upper airway in patients suffering from OSA. These devices, systems and methods for providing oral appliances can allow for the removal and adjustment of nasal dilators and tongue positioners.

Upper airway issues affecting respiration, including upper airway resistance, affects millions of individuals worldwide and can cause serious medical consequences, including co-morbidity with other health problems and death. Health problems due to upper airway issues may occur as a result of sleep disruption for individuals who snore and individuals who suffer from the associated problem of hypopnea during sleep.

Hypopnea is a condition characterized by shallow breathing or an abnormally low respiratory rate resulting in reduced air flow. Sleep apnea is a condition characterized by a temporary cessation of breathing that can result in complete cessation of air flow and thus suffocation. These upper airway issues can create conditions requiring the body of the affected individual to expend more effort than usual in order to overcome the reduced airflow. This can result in a fragmentation of sleep patterns due to awakening for short or extended periods of time. As such, these conditions often limit an individual's ability to enter deeper sleep stages that serve the vital function of refreshing and restoring the sleeping individual and are also damaging to many vital bodily systems and functions.

Additionally, temporomandibular joint and muscle disorders, commonly referred to as “TMJ” are known to cause sleeping problems and can be the cause of craniofacial pain in both children and adults. Various symptoms are associated with craniofacial pain including recurring and chronic headaches; earaches; ear stuffiness or ringing; neck pain or stiffness; facial pain; jaw joint clicking, popping or grating; limited ability to open or close the mouth; jaw locking in either open or closed positions; sensitive, loose or worn down teeth; pain or soreness in the temporomandibular joint; dizziness; pain or difficulty chewing or swallowing; pain behind the eyes; extreme sensitivity to light; attention-deficit hyperactivity disorder (ADHD); bed wetting and others.

The primary treatment for snoring, hypopnea, and apnea is the use of a device that is commonly referred to as a CPAP (Continuous Positive Air Pressure) device. These devices deliver pressurized air from a pumping component through a hose and into a mask that is secured over the nose of the individual. This has been one method of treating and correcting these problems.

Recently, treatment methods involving CPAP devices have been unsuccessful for a growing number of patients due to discomfort while using the devices, lifestyle issues, and portability problems associated with the devices when individuals travel. As a result, a significant number of patients have abandoned treatment only to experience worsening conditions and symptoms. Currently, there are few alternative treatment methods.

While surgical techniques have been available for many years, the attempt to permanently correct snoring problems encountered by individuals persists because surgical procedures are complicated and invasive. In some cases, surgical techniques leave permanent physical damage that effects the appearance of the patient. Additionally, surgical techniques involve other medical drawbacks including cost, lack of reversibility, risk of harm to the patient, and extensive and often painful recovery periods.

Various types of non-invasive devices have been developed in an attempt to alleviate or eliminate snoring, hypopnea and apnea issues. Some of these devices have focused on improving airflow through the nose. These devices are used by patients during vigorous activities that require increased airflow demand when they are awake and when asleep. This is one of the functions of an anti-snoring devices.

Some devices function by one of two mechanisms. The first mechanism requires attachment to the external skin of the nose on each side using adhesives. These devices pull the skin of the nose outward in order to strengthen and expand the nasal passages. These types of devices are disposable but can irritate the skin. The second mechanism requires the use of specialized devices that are designed to fit within the nasal passageway and push the inner walls of the nose outward in order to expand the air passageways. These types of devices can irritate the sensitive inner lining of the nasal passageway, the mucosa, and can be uncomfortable and awkward to use.

Other devices that attempt to alleviate or eliminate snoring problems without invasive surgery operate by repositioning the lower jaw or mandible in an anterior or forward position. This orientation can pull the base of the tongue forward within the mouth and thereby increase the size of the air passage in the posterior pharyngeal region, which is the breathing passage behind the base of the tongue. Devices that bring the mandible forward into as a functional repositioning posture and thereby hold the posterior airway open generally fall into two categories.

Included in the first category are the non-adjustable devices. These devices are placed within the mouth at a fixed, prescribed position. Non-adjustable devices have a significant disadvantage though. Over time, changes in anatomy or other conditions may occur altering placement and fit. As such, these devices may not be appropriate if there is a change in the position of the lower jaw with respect to the upper jaw.

The second category includes adjustable devices with components positioned inside the mouth and behind the teeth. These devices generally take up more space inside the mouth and restrict tongue space which prevents the tongue from moving forward. Some of these devices have projections that extend out of the mouth, between the lips. Disadvantages of these devices include the effect they have on a patient's ability to close their lips. This can make the device uncomfortable to wear and inhibit the patient's ability to change positions during sleep. As such, all current devices and techniques for improving air-flow have various drawbacks and disadvantages.

Some simple devices have recently been developed to treat sleep apnea. One such device is the OASYS system. This device developed by Dream Sleep Systems prevents the blockage of the throat by the tongue by holding the base of the tongue down and forward. However, such devices rely on materials that create a large amount of lingual bulk, which acts contrarily by forcing the tongue backward. This appliance also has attachments that are fixed to the patient's body. The inability to remove any of these attachments for modifications requires a complete recreation of the device. This is required also if the patient is not compliant with the modifications added. Another similar device used to treat sleep apnea is the EMA designed by Myerson company. The EMA does not offer or provide for nasal or lingual attachments.

When determining the most effective patient treatment, the practitioner may need to capture oral or other impressions of the patient's face and head. An accurate impression of the buccal sulcus is essential and needed for the correct positioning of the nasal dilators required in patient treatment, to open the patient's airway. This area is difficult to capture with impressions, especially digital impressions due to its location and discomfort to the patient. Without an accurate impression, the dental laboratory would find it extremely difficult or impossible to fabricate a therapeutic device with proper function.

Current oral appliance systems used for capturing digital impressions are generally unitary, and therefore not adjustable. As such, if a doctor or dentist wishes to adjust the oral appliance to a different position, the entire appliance must be sent to the dental laboratory to have the incorrect modification removed and a replacement fabricated. The patient is rarely present at the lab when the adjustments are being made to the replacement device. As such, the laboratory will estimate or guess what position the nasal dilator should be placed to provide the most effective and comfortable treatment. If the laboratory is incorrect in their assessment, the process of sending it from the dental practice back to the dental laboratory will repeat leading to patient and staff frustration. A modular system that allows a doctor to remove or adjust a nasal dilator without sending the entire system to a laboratory for re-fabrication would provide the best patient care outcome without cause for lapse in conditions.

Due to the varied nature of symptoms and potential negative results caused by snoring, hypopnea, sleep apnea, and TMJ, it would be desirable to treat these conditions using a non-invasive apparatus that does not suffer the drawbacks and disadvantages of current devices.

Thus, needs exist for improved techniques by which to allow for a nasal dilator to be removed and replaced with a different nasal dilator resulting in reduced costs, saved time, and the most effective patient treatment.

### SUMMARY

Provided herein are embodiments of systems, apparatuses and methods for manufacturing, building and using oral appliances for use with nasal dilators and tongue positioners.

Generally, these oral appliances allow users, such as doctors, RDA's, healthcare professionals, patients or other appropriate users to freely adjust a nasal dilator, the vertical position of the oral appliance, and tongue positioners associated with the oral appliance into a functional position. Various advantages provided by modular oral appliances for use with nasal dilators and lingual tongue positioners include improved comfort, convenience, functionality and success in preventing collapse of the airway.

To elaborate, they can be used to provide improved airflow through nasal and pharyngeal air passages, thus preventing snoring and sleep apnea. They can also be used as a single unit that works in a synergistic system to solve these problems. In some embodiments, they can provide a nasal and lingual breathing aid that acts intraorally and can be fixed in place by attachment to a device or removed depending on patient compatibility. Some embodiments include a nasal dilator that does not irritate or act as an obtrusive object in the buccal sulcus. Some embodiments can be used as an effective anti-snoring device that maintains maximum intraoral tongue space, as compared with previous devices. Some embodiments include a ring to function as a mandibular stop, preventing the jaw from falling back in supine position and therefore preventing the collapse of the airway. Various embodiments disclosed herein provide devices which are less bulky, more durable, and which are contained solely within the oral cavity. Some embodiments provide improved nasal breathing and tongue control that protects patients from traumatic injury and prevents them from developing a joint disorder. Some embodiments include a mandibular reposition device which can be used and incorporated by all medical professions treating Obstructive Sleep Apnea (OSA), Temporomandibular Joint Disorders (TMD), and snoring problems.

In various embodiments, flexible printed material can allow less bulky fabrication of oral appliances than previously developed devices that are equal if not more durable than previous devices. Examples of flexible materials that can be used include Nylon based materials such as PA2200. Since these materials are less bulky, they can maintain the tongue in a forward position while reducing airflow resistance through the pharyngeal region; preventing the tongue from falling backward in a supine position. This can be beneficial since the position of the tongue plays a large role in airway obstruction at rear positioned tongue regions.

Systems, methods, and apparatuses disclosed herein can also include an upper section from which one or more button or peg components extend. These buttons or pegs can extend, stretch or otherwise modify positioning of skin located on or near the upper lip and nose (buccal sulcus) regions for patients.

Upper and lower components, sections and pieces can be coupled or otherwise attached using one or more rubber bands, silicone bands, or other types of flexible bands. In various embodiments, bands can be composed of different shoreness and flexibility profiles, each having different characteristics, features, and benefits. This can allow physicians to monitor the nasal passage and help provide a simpler and more accurate determination as to whether the particular oral appliance nasal component in use is affecting the treatment in a positive manner. Some devices may include attachment rods or other components that are coupled or otherwise attached to each other in appropriate locations and fashions using materials that do not cause a “wear effect” or other use-based degradation. As such, these devices can provide direct feedback to the physician on a visual or physical basis.

In comparison with prior oral appliances that are used with nasal dilators, this current oral appliance provides wires with improved bendability. This can allow the oral appliance to provide improved functionality using interchangeable nasal dilators without requiring a new oral appliance to be manufactured each time a physician or patient wants to change the dilator. For example, shoreness features and qualities can be modified between soft, medium, and firm; and sizes can be modified between small, medium, and large in varying degrees. As such, a physician can select an appropriate dilator from a plurality of dilators, where each dilator has a certain degree of freedom of movement for correct positioning according to each individual patient's unique anatomy. Thus, physicians are better able to attend to and address the needs of each patient individually. Additionally, different nasal dilator sizes can be considered when selecting appropriate nasal dilators and a dilator can be easily exchanged if improper or undesired results are likely to occur, thus optimizing treatments on a per patient basis. As such, placement and adjustment are more efficient than prior devices, as is the ability to effectively treat individual users and their unique situations.

Those in the art will recognize that patients who may misplace their individualized oral appliance are able to have a new one fabricated quickly by a laboratory when using printed designs. This provides the benefit of reduced or eliminated requirements for medical professionals to create new records, saving time and money by improving efficiency. This also provides the benefit of allowing for manufacturing of replacement oral appliances that are substantially identical to their originals without requiring additional appointments to be scheduled or records to be created. Thus, patients are provided optimal comfort with minimal time requirements. Another benefit is the added advantage of time savings for medical professionals in the form of a reduction in time required to adjust these oral appliances and their associated nasal dilators when modifying or correcting positioning.

Based on the advantages of the modular designs disclosed herein, any damaged or destroyed components for nasal dilators can be replaced in the office by the professional. This removes the need to send the oral appliance back to a laboratory for adjustments. Thus, patients do not have to suffer unnecessarily without their oral appliances. If the patient is non-compliant, both the nasal dilators and tongue positions can be removed without lessening the integrity of the sleep device and without causing discomfort to the patient.

In various embodiments, the modular oral appliances described herein can be manufactured using three-dimensional (3D) printers and SLS machines. In some embodiments, one or more sleeves can be designed that is incorporated into an existing acrylic oral appliance.

The configuration of the devices described herein in detail are only example embodiments and should not be considered limiting. Other systems, devices, methods, features and advantages of the subject matter described herein will be or will become apparent to one with skill in the art upon examination of the following figures and detailed description. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE DRAWINGS

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 shows an example embodiment of an oral appliance from a front perspective view.

FIG. 2 shows an example embodiment of an oral appliance from an elevated side-perspective view.

FIG. 3 shows an example embodiment of an oral appliance from an elevated side-perspective view.

FIG. 4 shows an example embodiment of an oral appliance from an elevated side-perspective view.

FIG. 5 shows an example embodiment of an oral appliance from a side-perspective view.

FIG. 6A shows an example of an upper section oral appliance from a front lower perspective view.

FIG. 6B shows an example embodiment of a lower section of an oral appliance from a front lower perspective view.

FIG. 7 shows an example embodiment of an adjustable shim on a flat occlusal surface.

FIG. 8A shows an example embodiment of a locking mechanism for an adjustable shim.

FIG. 8B shows an example embodiment of an adjustable shim being bent to lock into an oral appliance.

FIG. 9A shows an example embodiment of a maxillary nasal pocket with a nasal channel from a perspective view.

FIG. 9B show an example embodiment of a negative of a maxillary nasal channel of a nasal pocket from a side view.

FIG. 9C show an example embodiment of a maxillary nasal pocket from the front view.

FIG. 10A shows an example embodiment of a maxillary portion of an oral appliance from an elevated proximal to distal top view.

FIG. 10B shows an example embodiment of a maxillary portion of an oral appliance from a top view.

FIG. 11A shows an example embodiment of a maxillary portion of an oral appliance from a cross-sectional front view.

FIG. 11B shows an example embodiment of a maxillary portion of an oral appliance from a front view, coupled with removable nasal dilators.

FIG. 12 shows an example embodiment of removable nasal dilators from a perspective view.

FIG. 13A shows an example embodiment of a lower section of an oral appliance including secondary coupling mechanisms from an elevated back-perspective view.

FIG. 13B shows an example embodiment of a lower section of an oral appliance including secondary coupling mechanisms from an elevated cross-sectional back-perspective view.

FIGS. 14A-14B show an example of coupling mechanisms for lingual tongue positioners of various sizes.

FIG. 15 shows an example embodiment of a lower section of an oral appliance from a top proximal to distal perspective view, including tongue positioners of varying dimensions.

FIG. 16 shows an example embodiment of interchangeable tongue positions of various sizes from a perspective view.

FIG. 17A shows an example embodiment of a band attachment button from a top perspective view.

FIG. 17B shows an example embodiment of a band attachment button from a side perspective view.

FIG. 18 shows an example embodiment of an oral appliance including a band attachment button from a side perspective view.

FIG. 19 shows an example embodiment of an oral appliance with a connecting band attached from an upper side-perspective view.

### DETAILED DESCRIPTION OF THE SYSTEM

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

Provided herein are systems, devices and methods for providing oral appliances that allow for adjustment of nasal dilators. In some embodiments, these appliances can be created or manufactured using a digital printer or SLS process. In some embodiments, a sleeve with a channel can be incorporated or added after manufacture to existing acrylic appliances.

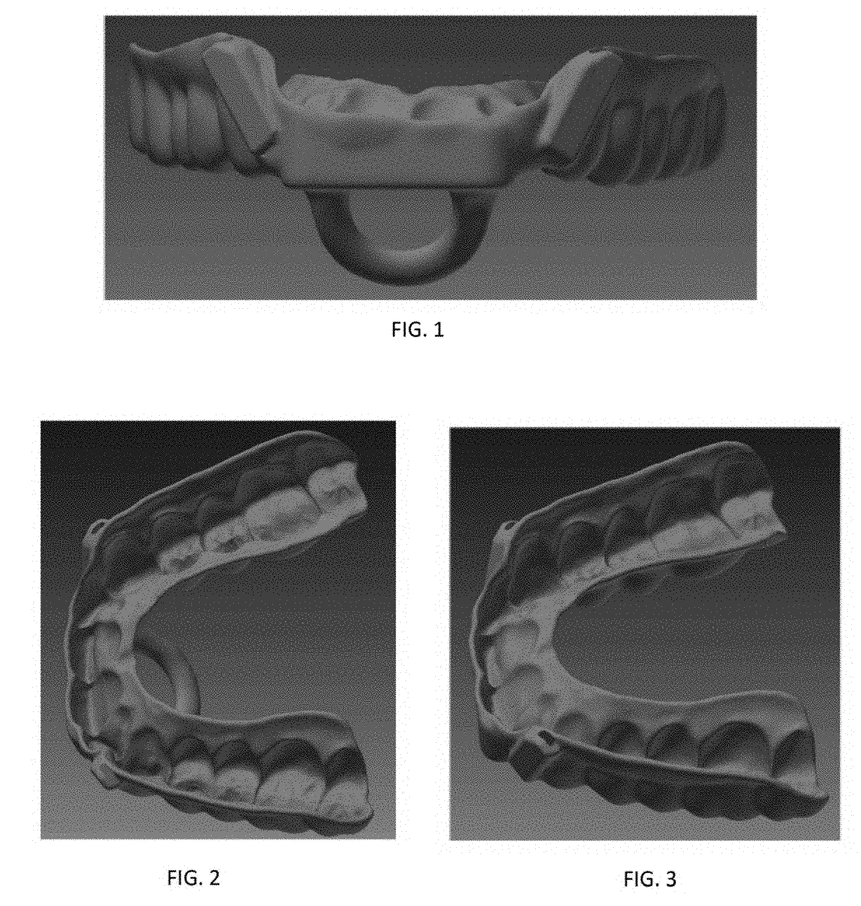

FIG. 1 shows an example embodiment of an oral appliance from a front perspective view. As shown in the example embodiment, an oral appliance can include a molding of a patient's teeth and a portion of surrounding oral geography. Moldings can be made in various manners and fashions as known in the art. One type of molding can include having a patient bite into a soft material before curing the material to fix the molding in position. Another type of molding can include having a patient bite into a soft material, removing the material and applying a second material to the first material and then curing the second material. Another type of molding can include applying a paste or other material to a patient's dental records and letting it cure. Another type of molding can include taking images of a patient's oral anatomy with a digital scanner or from impressions using a computer to map them before fabricating a molding, for instance by using digital software and SLS machinery. The ring prevents the patients jaw from retruding back during sleep or in supine position, therefore preventing collapse of the airway.

FIG. 2 shows an example embodiment of an oral appliance from an elevated side-perspective view. As shown in the example embodiment, a channel can be located on a first side of an oral appliance. The first channel can have a rectangular, square, v-shaped, semicircular or other cross sectional profile with one or more walls creating sides of the channel. The channel can be enclosed except for an upper and lower end, as shown or can have an open side or sides. The channel shown generally has a slope of about 45-60 degrees from a plane located across the teeth and is directed such that it slopes toward a center of the mouthpiece. The channel generally begins in front of canine or incisor and ends in front of a front tooth. The channel is located on a frontal surface of the mouthpiece, exterior to a tooth bed pointing in the direction of the buccal sulcus.

FIG. 3 shows an example embodiment of an oral appliance from an elevated side-perspective view. As shown in the example embodiment, a second channel can be similarly sized and situated, except in a complementary location opposite the first channel and on the front surface of the mouthpiece, exterior to a tooth bed.

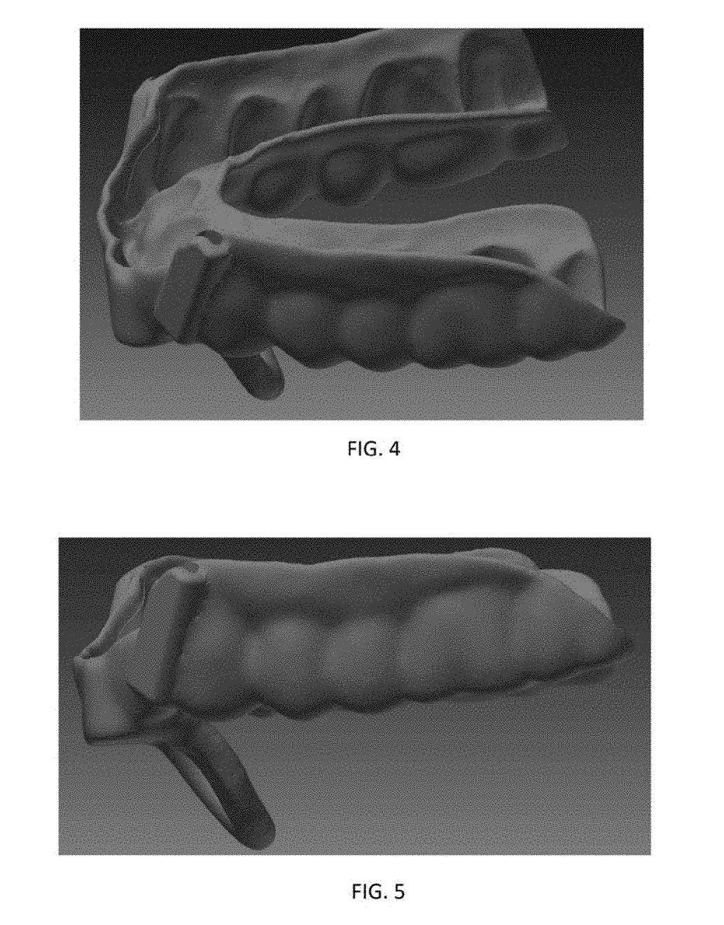

FIG. 4 shows an example embodiment of an oral appliance from an elevated side-perspective view. As shown in the example embodiment, a loop or partial loop can be provided on an underside of the mouthpiece. In the example embodiment, the loop can be formed by a length of material that has a cross sectional profile that is circular. The loop is bent such that it creates a u-shaped protuberance below the mouthpiece. As such, when in a patient's mouth, the loop will rest just behind the lingual region of the lower anterior teeth. The loop is generally large enough such that it gives space for the tongue to sit, as well as increase airflow. The loop can be formed with the same material as the mouthpiece itself or it can be formed from a different material. The loop may be flexible, semi-flexible or rigid in various embodiments.

FIG. 5 shows an example embodiment of an oral appliance from a side-perspective view. As shown in the example embodiment, a wire can be placed or routed through one channel, opening the semi-circular bend increases and decreases the retention of the wire in the nasal pocket. As such, the wire can maintain the hose or item in a fixed or semi-fixed position with respect to the mouthpiece. This can help prevent undesired movement of the hose or item. It should be understood that one or both ends of the wire can be anchored in place using a stopping mechanism.

Also, as shown in the example embodiment, at least a portion of the mouthpiece located in front of the teeth can have a substantially flat surface such that it acts as a platform for the lower portion to occlude without interferences or concern of tooth movement.

In some embodiments, instead of a sleeve, a raised or otherwise elevated bar or rail can be positioned to allow a printed button to slide over the top of the oral appliance and be held in place. This can allow for oral appliances to be designed and fabricated completely digitally using a computer and computer directed equipment that is automated. This can also allow for replacement buttons that may be fabricated such that they are an exact match with originals.

In some embodiments, the principles and concepts described herein can also be used for designing and creating tongue lift attachments.

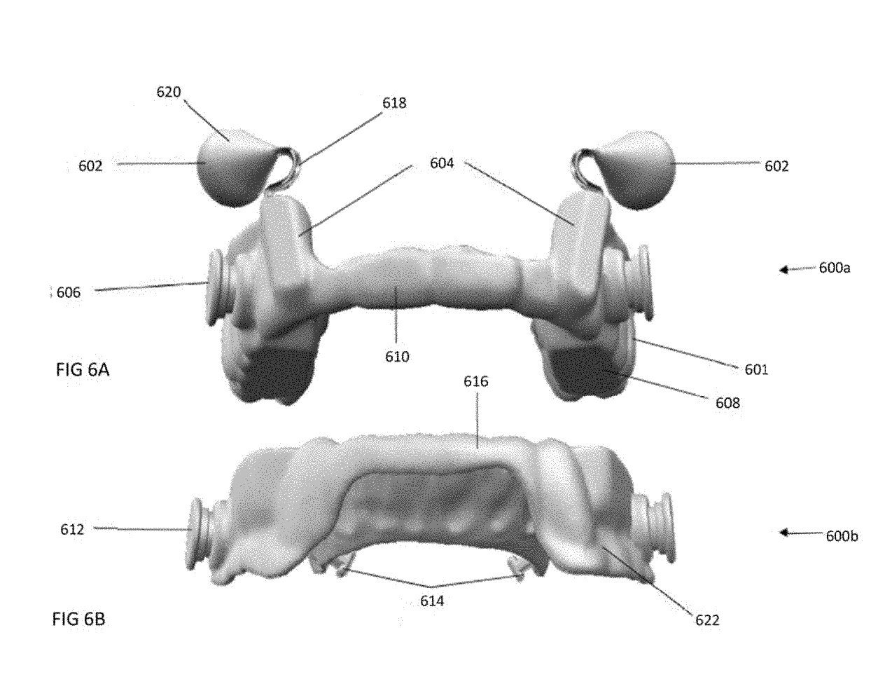

FIG. 6A shows an example of an upper section 600a of an oral appliance from a front lower perspective view. As shown in FIG. 6A, upper section 600a can include an upper section body 601 that provides a frame which other components can be permanently or removably coupled with or mounted to in various embodiments. Upper section body 601 is generally a casting mouthpiece of a patient's maximal, superior, or upper dental arch. Upper section body 601 can include a custom-molded mouthpiece 610 that includes at least one flat occlusal surface 608 of the upper section 600a. This flat occlusal surface can be approximately located below a portion of all of the area between a second bicuspid and a first molar along both extensions, arms, or sides of upper oral appliance 600a and positioned facing downward, toward the inferior, mandibular or lower dental arch. As such, flat occlusal surface 608 can function as a posterior vestibular support pad. In some embodiments, flat occlusal surface 608 can provide the patient with occlusion that is free from occlusal or other forces during operation. In some embodiments flat occlusal surface 608 can be configured to accept an adjustable shim to adjust the vertical configuration of upper section 600a. In some embodiments, upper section 600a of an orthotic can include one or more discluding elements, components, or systems. In some embodiments, upper section 600a of an orthotic can include one or more or anterior deprogrammer elements, components, or systems. These elements, components, or systems may allow a patient's temporomandibular joint to decompress, while also treating problems or issues related to the patient's airway.

One or more maxillary nasal pockets 604 can be included in or formed along with upper section body 601 during manufacturing. Alternatively, nasal pockets 604 can be permanently or removably affixed, attached, connected, or otherwise coupled with body 601 in a post manufacturing process. Nasal pockets 604 generally include one or more hollow channels, formed within an exterior casing. Channels can be open at one or more locations. In the example embodiment, channels are open at an upper location of the device and the openings are located between a patient's upper lip and gums. Channels can be positioned substantially vertically or at various angles and can include securing components in various locations in some embodiments. As shown in the example embodiment, one or more nasal pockets 604 can be designed to hold or maintain a nasal dilator 602 in a fixed or minimally movable position.

As shown in the example embodiment, one or more nasal dilators or nasolabial dilators 602 can be coupled, attached, or otherwise connected to maxillary nasal pocket 604 of the upper section 600a of a body of the oral apparatus or orthotic. This can push the upper lip into a position that is located away and spaced apart from a patient's maxillary dental arch, thereby dilating the nasal passage of the patient, improving airflow through nasal-pharyngeal airways. As shown in the example embodiment, a wire 618 of nasal dilator 602 can be permanently or removably coupled to a bulb 620 of nasal dilator 602. Bulb 620 can have different regular or irregular shapes including cylindrical, conical, spherical, or various others as well as materials with different shore hardness and rigidity.

As also shown in the example embodiment, an upper section 600a can include at least one coupling mechanism 606, referred to herein as a peg or button. Coupling mechanism 606 can be included in or formed along with upper section body 601 during manufacturing. Alternatively, coupling mechanism 606 can be permanently or removably affixed, attached, connected, or otherwise coupled with body 601 in a post manufacturing process. Coupling mechanism 606 can be generally cylindrical, extend perpendicular to an exterior gum-facing surface of body 601 and have one or more ridges a distal edge, perpendicular to a central axis of the cylindrical body. In some embodiments, coupling mechanism 606 can be used to secure upper section 600a with a lower section of a multi-section oral appliance, for example as shown and described with respect to lower section 600b of FIG. 6B.

In some embodiments, upper section body 601 can be composed of or otherwise made or manufactured from one or more materials that can be removably coupled with the patient's maximal dental arch. These materials can be homogeneous or heterogeneous compositions and can be all or portions of body 601.

Examples of processes for manufacturing body 601 using appropriate and effective materials include using a processed acrylic that has a hard-molded outer shell and, optionally may include a soft inner lining, to capture the patient's maximal dental arch when coupled with the maximal dental arch in an appropriate position and fashion. Manufacturing processes can also include the use of “boil-and-bite” materials, pre-formed or pre-fabricated common arch forms, or other processes and materials currently known in the art or later developed. In some embodiments, body 601 can be constructed of a thermoplastic material, such as BIOCRYL available from Great Lakes Orthodontics, which is heat molded over a dental model of the patient's teeth. To elaborate, 3 mm BIOCRYL may be pressure molded over a dental cast of the maximal dental arch area using a BIOSTAR® thermoplastic molding unit, also available through Great Lakes Orthodontics. This process of molding can include taking images of a patient's maximal oral cavity and using a computer to map its various features before fabricating a molding. Fabrication of moldings can be performed using a 3D printer and associated software in some embodiments. Intraoral scanners can allow patient information to be captured and stored in digital format before being transmitted or otherwise delivered to a 3D printer device for fabrication.

FIG. 6B shows an example embodiment of a lower section 600b of an oral appliance from a front lower perspective view. Lower section 600b can include a body 622 having a custom-molded mouthpiece 616 of a patient's inferior dental arch and may include other features of the patient's inferior oral cavity.

As also shown in the example embodiment, a lower section 600b can include at least one coupling mechanism 612, referred to herein as a peg or button. Coupling mechanism 612 can be included in or formed along with lower section body 622 during manufacturing. Alternatively, coupling mechanism 612 can be permanently or removably affixed, attached, connected, or otherwise coupled with body 622 in a post manufacturing process. Coupling mechanism 612 can be generally cylindrical, extend perpendicular to an exterior gum-facing surface of body 622 and have one or more ridges a distal edge, perpendicular to a central axis of the cylindrical body. In some embodiments, coupling mechanism 622 can be used to secure lower section 600b with an upper section of a multi-section oral appliance, for example as shown and described with respect to upper section 600a of FIG. 6A.

As shown in the example embodiment, one or more secondary coupling mechanisms 614 can couple with associated lingual tongue positioners for use by patients. Secondary coupling mechanisms 614 can be “T” shaped extensions in some embodiments that are permanently or removably coupled with body 622 in locations toward the rear of the device and may be positioned substantially horizontally or slightly angled in a downward position.

Examples of processes for manufacturing body 622 using appropriate and effective materials include using a processed acrylic that has a hard-molded outer shell and, optionally may include a soft inner lining, to capture the patient's inferior dental arch when coupled with the inferior dental arch in an appropriate position and fashion. Manufacturing processes can also include the use of “boil-and-bite” materials, pre-formed or pre-fabricated common arch forms, or other processes and materials currently known in the art or later developed. In some embodiments, body 622 can be constructed of a thermoplastic material, such as BIOCRYL available from Great Lakes Orthodontics, which is heat molded over a dental model of the patient's teeth. To elaborate, 3 mm BIOCRYL may be pressure molded over a dental cast of the inferior dental arch area using a BIOSTAR® thermoplastic molding unit, also available through Great Lakes Orthodontics. This process of molding can include taking images of a patient's inferior oral cavity and using a computer to map its various features before fabricating a molding. Fabrication of moldings can be performed using a 3D printer and associated software in some embodiments. Intraoral scanners can allow patient information to be captured and stored in digital format before being transmitted or otherwise delivered to a 3D printer device or SLS machine for fabrication.

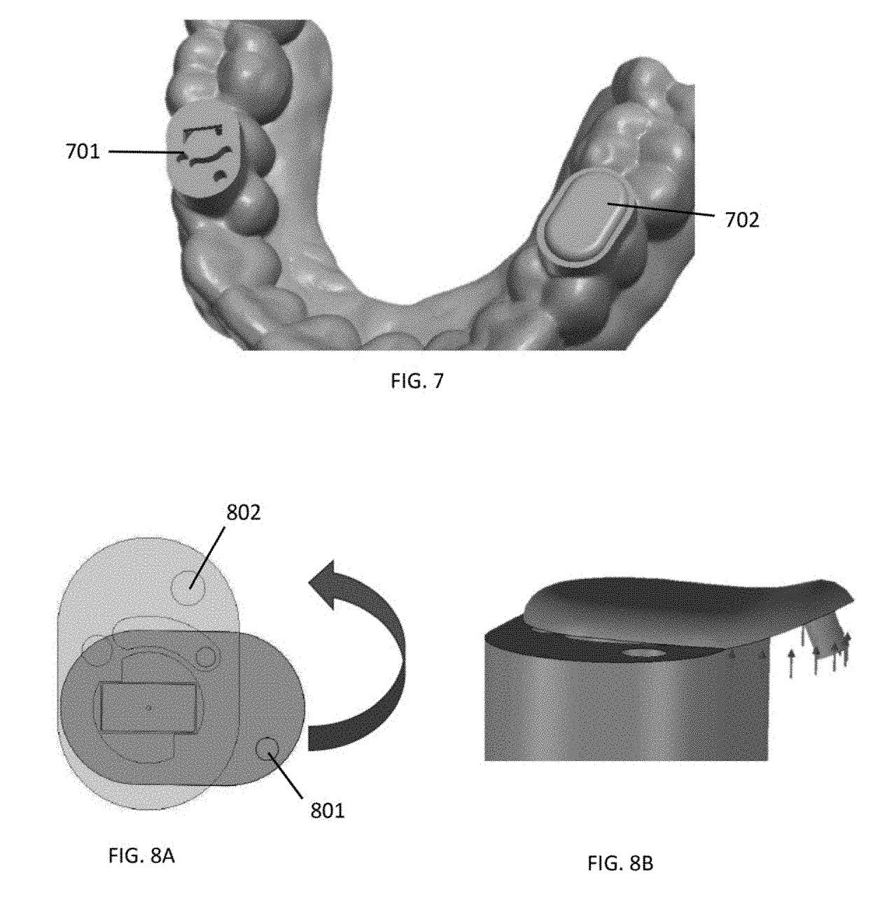

FIG. 7 shows an example embodiment of an adjustable shim 702 on a flat occlusal surface 608. As shown in the example embodiment, the flat occlusal surface 608 is configured to lock in the adjustable shim through the use of specially designed cut outs 701 that allow the adjustable shim 702 to turn and lock into place on the flat occlusal surface 608. The specially designed cut outs 701 can be placed on a flat occlusal surface on either upper section 600a or lower section 600b. The flat occlusal surface 608 and specially designed cut outs 701 could also be placed at the anterior or front of the custom mouthpiece 610.

FIG. 8A shows an example embodiment of a locking mechanism for an adjustable shim.

FIG. 8B shows an example embodiment of an adjustable shim 702 being bent to lock into an oral appliance.

As shown in FIGS. 8A-8B, the adjustable shim 702 can be turned ninety degrees after being inserted into the flat occlusal surface 608, which allows the adjustable shim 702 to be locked into a fixed position using pegs. In an example embodiment, adjustable shim 702 is constructed of a flexible material such as nylon so the adjustable shim 702 can be bent, allowing peg 801 to clear the edge of the flat occlusal surface 608 and be inserted into hole 802.

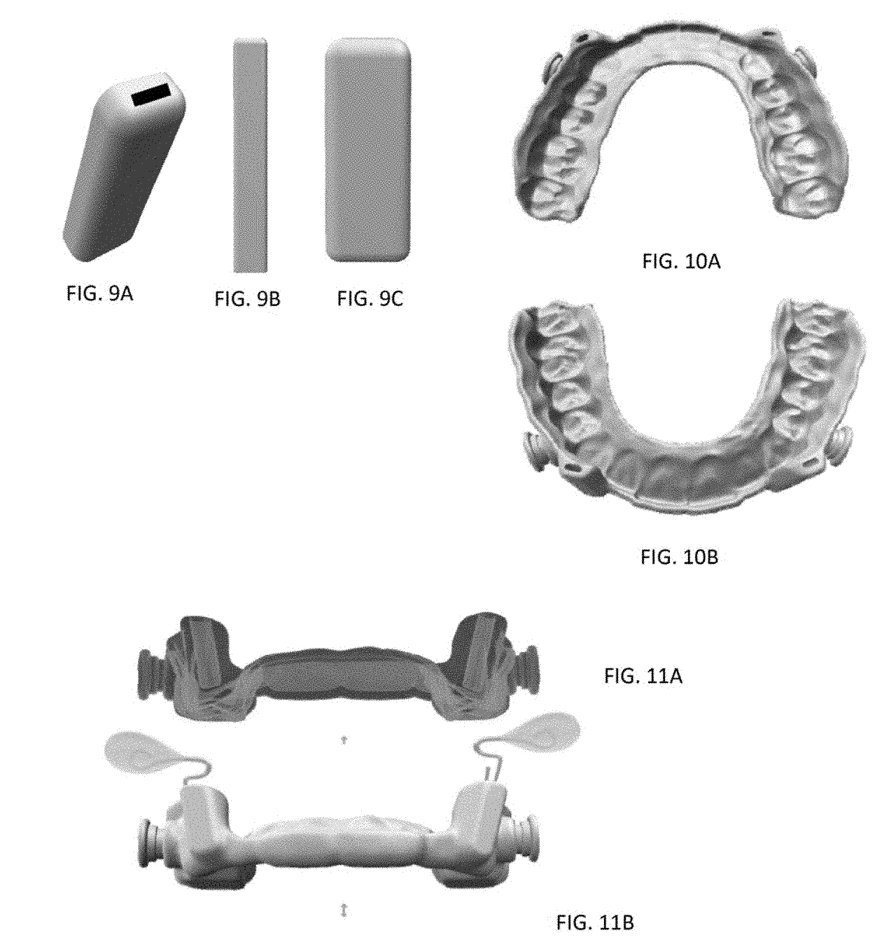

FIG. 9A shows an example embodiment of a negative of a maxillary nasal pocket channel of a nasal pocket from a perspective view. This pocket can be analogous to maxillary nasal pocket 604 of FIG. 6A in some embodiments. As shown in the example embodiment, the negative is a cut out designed to remove material from the nasal dilator pocket to allow the connecting wire to be inserted. This dimensions of the nasal pocket channel can be generally constant in some embodiments. In an example embodiment, it can be shaped to receive wire of about 0.036 mm diameter. The channel may generally have a slope of between about 45 degrees to 60 degrees from a plane located across the teeth and may be positioned such that it slopes downward toward a central location of the upper oral appliance between the central incisors and angles upward toward the buccal sulcus. The channel generally begins in front of canine or incisor and ends in front of a lateral incisor facing the buccal sulcus. The channel is located on a frontal surface of the mouthpiece, exterior to a tooth bed.

FIG. 9B show an example embodiment of a negative of a maxillary nasal pocket channel of a nasal pocket from a side view. This is the portion removed from the nasal dilator buildup to allow the connecting wire to house the nasal dilator.

FIG. 9C show an example embodiment of a negative of a maxillary nasal pocket channel of a nasal pocket from a top view.

FIG. 10A shows an example embodiment of a maxillary portion of an oral appliance from an elevated proximal to distal top view.

FIG. 10B shows an example embodiment of a maxillary portion of an oral appliance from a top view.

As shown in FIGS. 10A-10B, maxillary sections of oral appliances generally include nasal dilator pockets having channels. The size of the channels or holes can be constant, where walls of the channel provide frictional resistance to a nasal dilator wire inserted in the channel to prevent the nasal dilator from becoming dislodged from the body of the maxillary portion or section of the oral appliance.

FIG. 11A shows an example embodiment of a maxillary portion of an oral appliance from a cross-sectional front view showing the nasal dilator pockets extending from the most superior portion of the device to the most inferior portion.

FIG. 11B shows an example embodiment of a maxillary portion of an oral appliance from a front view, coupled with removable nasal dilators. As shown in the example, nasal dilators can be coupled with the base of the upper unit that are operable to be used to dilate a patient's airway. As shown, the wire can be placed or routed through at least one channel of the maxillary section of the oral apparatus. In various embodiments, nasal dilator structures are modular and include at least one bulb structure and at least one wire structure. Bulbs can be generally planar in some embodiments, tear-drop shaped, or others as described herein or appropriate.

Modular nasal dilator structures can be fabricated with various appropriate materials with different properties, such as soft, medium, and hard firmness. Examples of appropriate materials are BIOCRYL ICE from Great Lakes Orthodontics and other forms of methacrylate materials. Wires can be the same material or different material from bulbs of the nasal dilators. Dilator bulbs can be rotated and repositioned by manipulating and changing the angle of the wires, which can include wire stem bending as shown. This bending can be fixable in some embodiments and allow for movement and fixation anteriorly, posteriorly, laterally, and in other directions. As such, wired can maintain any hoses, attachments, or other items being used in conjunction with the oral apparatus in a fixed or semi-fixed position with respect to the mouthpiece, even if the patient moves. These features help to prevent or minimize any undesired movement of the hoses, attachments, or other items with respect to the body of the maxillary section of the oral apparatus. As such, patients and physicians are able to freely move, adjust, remove, and otherwise adjust or manipulate the hoses, attachments, or other items as necessary. It should be understood that one or both ends of the wire can be anchored in place using a fixed or movable stopping mechanism. Examples include, ledges, ridges, latches, flaps, and others as appropriate.

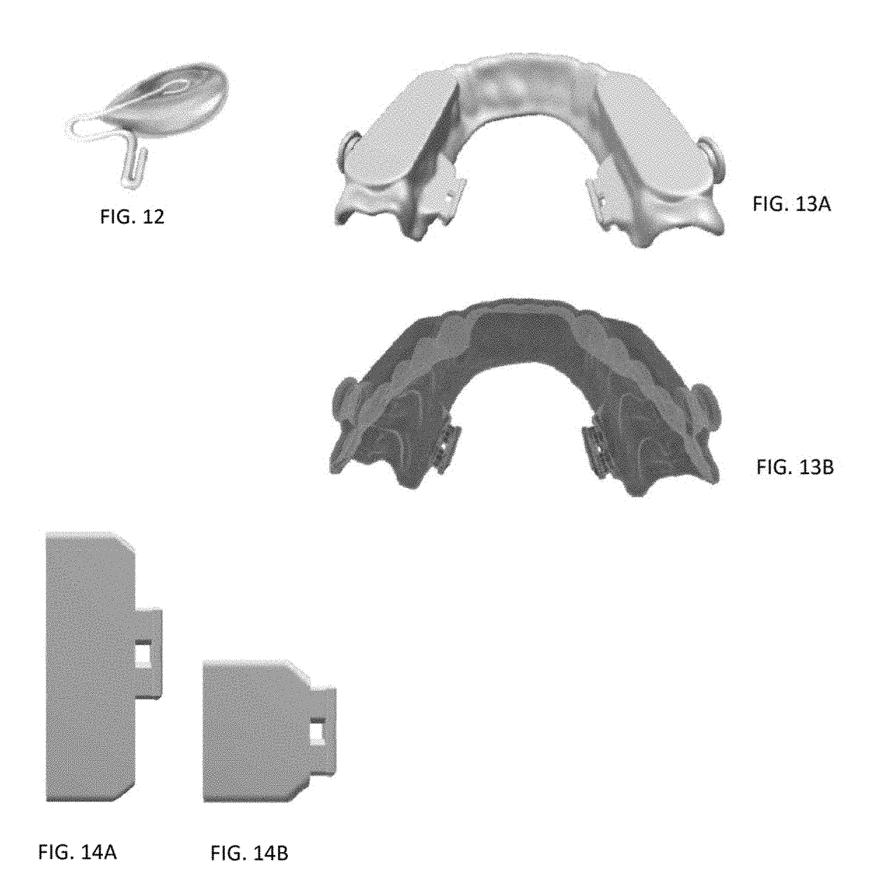

FIG. 12 shows an example embodiment of fixed or removable nasal dilators from a perspective view. Bulbs of nasal dilators are coupled with one end of nasal dilator wires, which can be bent in a variety of orientations during manufacturing or treatment for various purposes. In some embodiments, channel resistance to movement of the wire can be increased or decreased by modifying, manipulating, or moving one or more bends or loops of the wire closer together or further apart. Similarly, modifying, manipulating, or moving bends or loops of the wire can allow the bulb to provide increased or decreased resistance to lip, nasal, or other nearby structures when adjusted. The maneuverability and manipulability of wires allows these nasal dilators to be secured with respect to upper sections of oral appliance apparatuses if the patient is compliant with the position of the nasal dilator. Likewise, maneuverability and manipulability of wires allows these nasal dilators to be removed with respect to upper section channels of oral appliance apparatuses if necessary to adjust for improved functionality or replacement.

FIG. 13A shows an example embodiment of a lower section of an oral appliance including secondary coupling mechanisms for lingual tongue positioners from an elevated back-perspective view.

FIG. 13B shows an example embodiment of a lower section of an oral appliance including secondary coupling mechanisms for lingual tongue positioners from an elevated cross-sectional back-perspective view. This shows the locking mechanism in the middle of the secondary coupling mechanism where the tongue positioner slides and locks in.

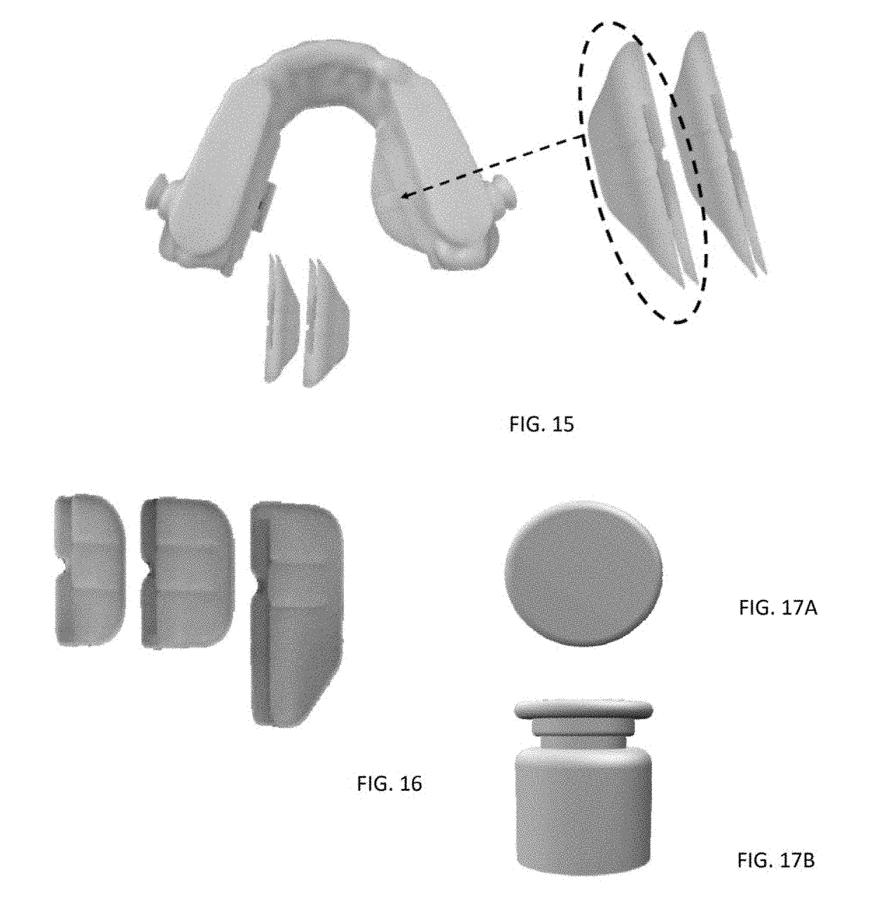

As shown in the example embodiment of FIGS. 13A-13B, secondary coupling mechanisms can provide lingual tongue positioner connection or coupling locations that help to position and pull the base of a patient's tongue forward when coupled with lingual tongue positioners. Example embodiments of lingual tongue positioners are further shown and described with respect to FIG. 15. Secondary coupling mechanisms to secondary coupling mechanisms 614 of FIG. 6B and are also further described with respect to FIGS. 14A-14B.

FIGS. 14A-14B show an example of coupling mechanisms for lingual tongue positioners of various sizes. As shown in the example embodiment, a mechanism for coupling, connecting or otherwise attaching lingual tongue positioners can include a slot with parallel and opposing sides that slides over and secures with secondary coupling mechanisms of a lower section of an oral appliance. In various embodiments, different sizes, shapes, and types of features for lingual tongue positioners can be used, as can different coupling, attaching, and connecting mechanisms. These various embodiments provide physicians and patients the ability to select lingual tongue positioners that are comfortable, while providing optimal effectiveness for individual patients with unique inferior dental arch anatomical structures.

FIG. 15 shows an example embodiment of a lower section of an oral appliance from a top proximal to distal perspective view, including tongue positioners of varying dimensions. As shown in the example embodiment, a lower section of an oral appliance can be a mandibular portion of a multi-section oral appliance system or device. Secondary coupling mechanisms as shown in the example embodiment can allow one or more lingual tongue positioners to be coupled with the body of the lower section of the oral appliance device. Secondary coupling mechanisms can be varied in different embodiments and in the example embodiment include a “T” shape extension coupled with the body of the lower section of the oral appliance. This T section can be oriented such that the cross at the top is substantially parallel to the oral appliance device from which the perpendicular bottom section of the T extends. This top of the T section is referred to herein as an interior ridge. The interior ridge is operable to support and maintain a position lingual tongue positioners, as shown.

In some example embodiments, lingual tongue positioners can include a slot in at least one side that is sized such that it is operable to slidably receive and engage the interior ridge of the secondary coupling mechanism. One or more extensions in the slot can removably mate with a comparably sized compatible depression in the secondary coupling mechanism.

Lingual tongue positioners can be various sizes that are able to accommodate different inferior oral anatomy arrangements comfortable for patients. In some embodiments, lingual tongue positioners can be coupled with secondary coupling mechanisms in different orientations. In some orientations, lingual tongue positioners can be coupled in an orientation that is angled between about 45 degrees to 60 degrees above a horizontal plane. In some orientations, this can be inverted, such that they are coupled in an orientation that is angled between about 45 degrees to 60 degrees below a horizontal plane. Other angles are also contemplated, and may be adjusted according to a patient's anatomical arrangements. Similarly, secondary coupling mechanisms of the oral appliance body can have differing orientations as well.

Lingual tongue positioners can affect tongue positioning in the mouth and thereby improve patient respiration by repositioning the jaw and tongue in an anterior position, as compared with a normal or standard position. This can increase the area available for air passage in the posterior pharyngeal region. Since positioning lingual tongue positioners and secondary coupling components in this location in appropriate orientations can open the breathing passage behind the tongue, the embodiments of lower sections of oral appliance apparatuses described herein can help doctors to better alleviate snoring and other sleep related problems for their patients, as compared with current non-invasive treatment methods and invasive treatment methods including surgery.

FIG. 16 shows an example embodiment of interchangeable tongue positions of various sizes from a perspective view. As shown, lingual tongue positioners can have a wall at one end of the slot that helps to maintain the lingual tongue positioner in a substantially fixed position with respect to the body of a lower section of an oral appliance.

FIG. 17A shows an example embodiment of a band attachment button from a top perspective view. As shown in the example embodiment, the body can be generally cylindrical with one or more circumferential grooves or ridges to maintain a band in position with respect to the device. Buttons can be oriented outward with respect to oral appliance bodies as shown in 606 of FIG. 6A and 612 of FIG. 6B. Bands can be removably coupled with buttons on upper and lower sections of oral appliances in order to fix their position in a generally fixed position with respect to each other. One or more buttons can be removably or fixedly coupled with the section bodies. In some embodiments, bands can be elastic to allow for minimized movement or retrusion of the jaw. In other embodiments, bands can be more rigid.

FIG. 17B shows an example embodiment of a band attachment button from a side perspective view.

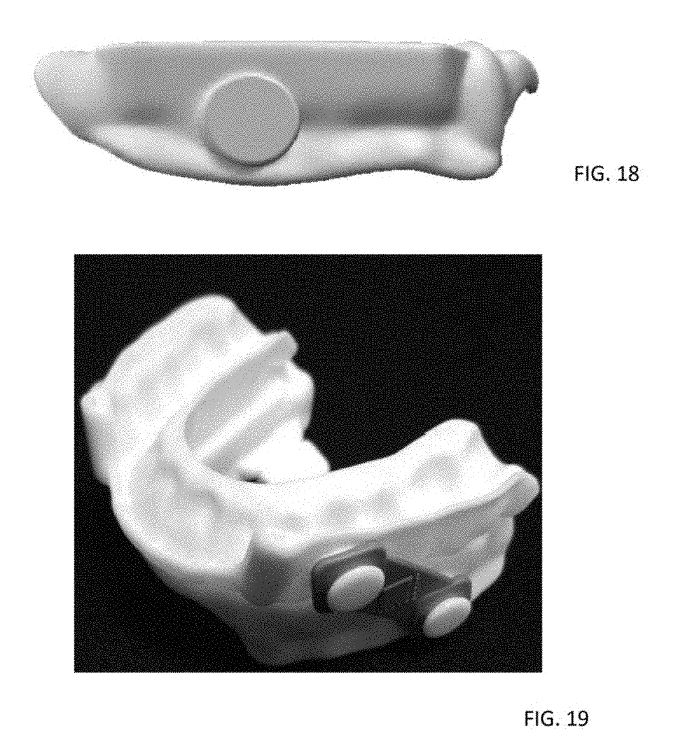

FIG. 18 shows an example embodiment of a lower section of an oral appliance including a band attachment button from a side perspective view.

FIG. 19 shows an example embodiment of an oral appliance with a connecting band attached from a upper side-perspective view. As shown in the example embodiment, the band can be a rigid mechanical structure with an elongated body and receiving holes to secure to buttons on upper and lower sections of an oral appliance.

As shown in the example embodiment, buttons, hooks, and other protrusions can be coupled with or attached to the side of an oral appliance upper and lower section mouthpiece that may be used to removably couple or connect the upper and lower sections together and provide adjustability. In some embodiments, bands can be composed of one or more materials or compositions such as silicone, elastosil, rubber, and others that provide some amount of flexibility. In some embodiments these bands may be produced with square fastening loops with rounded corners to reduce the rubbing on a patient's gums and gum line.

Different wear rates can be provided for with different materials and compositions. As such, bands can provide doctors or physicians to monitor use of the bands as a feedback device. If patients are wearing through bands rapidly or frequently breaking them, this can indicate that additional or other components may require adjustment or analysis, for example, a nasal dilator component. The channel in the body of the upper sections provides the ability for nasal dilators to be inserted or removed at any time without interfering with the device or the patient's course of treatment.

In some embodiments, the principles and concepts described herein can also be used for designing and creating tongue lift attachments.

As used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together, or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

Thus, a system for an oral appliance device has been described.

What is claimed is:

1. An oral appliance apparatus, comprising:
a mouthpiece fit for use by a human around a plurality of teeth;
at least one channel molded in front of at least one tooth and oriented vertically within the human's mouth when in use; and
a loop molded in the mouthpiece having a substantially rigid body and operable to maintain an opening and prevent mandible from dropping back in supine position therein.

2. An oral appliance apparatus, comprising:
a removable mouthpiece having an upper body and lower body fit for use by a human around a plurality of teeth.

3. The oral appliance apparatus of claim 2, further comprising:
an upper body primary coupling mechanism coupled with the upper body; and
a lower body primary coupling mechanism coupled with the lower body.

4. The oral appliance apparatus of claim 3, further comprising:
a removable fastener, operable to removably couple with the upper primary coupling mechanism and the lower primary coupling mechanism.

5. The oral appliance apparatus of claim 2, further comprising:
a secondary coupling mechanism coupled with the lower body.

6. The oral appliance apparatus of claim 5, further comprising:
a removeable tongue positioner, operable to be removably coupled with the secondary coupling mechanism.

7. The oral appliance apparatus of claim 2, further comprising:
a nasal pocket coupled with the upper body.

8. The oral appliance apparatus of claim 7, further comprising:
a nasal dilator, operable to be removably coupled with the nasal pocket

9. The oral appliance apparatus of claim 8, wherein the nasal dilator further comprises:
a wire; and
a bulb.

10. The oral appliance apparatus of claim 2, further comprising:
where the vertical distance between the upper body and lower body can be adjusted.

11. The oral appliance apparatus of claim 5, further comprising:
where the vertical distance between the upper body and lower body can be adjusted.

12. The oral appliance apparatus of claim 6, further comprising:
where the vertical distance between the upper body and lower body can be adjusted.

13. The oral appliance apparatus of claim 7, further comprising:
where the vertical distance between the upper body and lower body can be adjusted.

14. The oral appliance apparatus of claim 8, further comprising:
where the vertical distance between the upper body and lower body can be adjusted.
